(19) United States
(12) Patent Application Publication
(10) Pub. No.: US 2026/0207224 A1
(43) Pub. Date: Jul. 23, 2026

(54) IN VITRO FERTILIZATION (IVF) EMBRYO TRANSFER CATHETER

(71) Applicant: Regents of the University of Minnesota, Minneapolis, MN (US)

(72) Inventors: Daniel Martin BROBERG, Minneapolis, MN (US); Christian ANGELOPOULOS, Saint Louis Park, MN (US)

(73) Assignee: Regents of the University of Minnesota, Minneapolis, MN (US)

(21) Appl. No.: 19/134,065

(22) PCT Filed: Dec. 20, 2023
(86) PCT No.: PCT/US2023/085130

§ 371 Date: May 29, 2025

Related U.S. Application Data
(60) Provisional application No. 63/434,372, filed on Dec. 21, 2022.

(51) Int. Cl. A61B 17/435; A61B 90/00; A61M 25/00; A61M 25/01; A61M 25/10

(52) U.S. Cl. CPC A61B 17/435; A61M 25/0108; A61M 25/10; A61B 2090/3925; A61M 2025/0004; A61M 2202/0007

(57) ABSTRACT

A catheter assembly for embryo transfer during in vitro fertilization (IVF) procedures is disclosed. The catheter assembly comprises an elongated inner catheter shaft comprising a proximal end, a distal end, and a central lumen. The steerable catheter assembly further comprises an axially movable plunger extending at least partially within the central lumen. The plunger is configured to axially move a first distance within the central lumen and to positively displace a volume of a fluid in the central lumen by about the first distance.

### CROSS-REFERENCE TO RELATED APPLICATIONS

This application claims priority to U.S. Provisional Application Number 63/434,372 that was filed on Dec. 21, 2022. The entire content of the applications referenced above is hereby incorporated by reference herein.

### FIELD

The present application generally relates to medical devices and, in particular, to a catheter for controlled placement of an embryo at a target location within a patient's uterus.

### BACKGROUND

In Vitro Fertilization (IVF) is a widely used procedure used to treat patients who are not able to naturally conceive a pregnancy. Placement of an embryo in the patient's uterus is the last step of the IVF procedure, following egg harvesting, retrieval, and fertilization in the laboratory. Accuracy and precision are key to successful implantation of the embryo at a target location within the uterus. A misplaced embryo can result in a potentially lethal ectopic pregnancy, in which a fertilized egg implants and grows outside the main cavity of the uterus, most often in one of the fallopian tubes. The risk of a misplaced embryo compounds the already costly, time-consuming, and stressful process of IVF, and it can be attributed to the shortcomings of existing embryo transfer catheters. Thus, there is a need for a device that provides for controlled, accurate, and precise placement of an embryo, resulting in safe and successful implantation within the uterus.

### BRIEF SUMMARY

A catheter assembly for embryo transfer during in vitro fertilization (IVF) procedures is disclosed. The catheter assembly comprises an elongated inner catheter shaft comprising a proximal end, a distal end, and a central lumen. The steerable catheter assembly further comprises an axially movable plunger extending at least partially within the central lumen. The plunger is configured to axially move a first distance within the central lumen and to positively displace a volume of a fluid in the central lumen by about the first distance.

Accordingly, certain embodiments provide a catheter assembly comprising: an elongated inner catheter shaft comprising a proximal end, a distal end, and a central lumen; and an axially movable plunger extending at least partially within the central lumen; wherein the plunger is configured to axially move a first distance within the central lumen and to positively displace a volume of a fluid in the central lumen by about the first distance.

In certain embodiments, the catheter assembly further comprises a seal between the plunger and the central lumen.

In certain embodiments, the plunger is configured to move proximally to draw fluid from outside the inner catheter shaft through the distal end and into the central lumen.

In certain embodiments, the fluid is non-compressible.

In certain embodiments, the fluid is culture media comprising an embryo.

In certain embodiments, the plunger is configured to move distally to transfer the embryo from the central lumen out through the distal end of inner catheter shaft.

In certain embodiments, the plunger is configured to move distally to transfer the embryo from the distal end of the inner catheter shaft to a target location within a uterine cavity.

In certain embodiments, an outer diameter of the plunger is approximately equal to an inner diameter of the central lumen.

In certain embodiments, the plunger comprises PTFE.

In certain embodiments, the catheter assembly further comprises a drive wheel frictionally mated with the plunger; wherein rotational movement of the drive wheel translates to linear movement of the plunger.

In certain embodiments, the drive wheel is configured to be operated manually, via a motor, or robotically.

In certain embodiments, the catheter assembly further comprises one or more markers visible via ultrasound.

In certain embodiments, the catheter assembly further comprises an elongated outer catheter shaft, wherein the inner catheter shaft is configured to be inserted into the outer catheter shaft.

In certain embodiments, the catheter assembly further comprises at least one pull wire extending within a wall of the outer catheter shaft from a proximal end of the outer catheter shaft to a distal end of the outer catheter shaft; wherein the at least one pull wire is configured to deflect the distal end of the outer catheter shaft.

In certain embodiments, the catheter assembly further comprises at least one expandable balloon axially mounted to a distal end of the outer catheter shaft.

In certain embodiments, the catheter assembly further comprises a visualization catheter shaft configured to be inserted into the outer catheter shaft.

In certain embodiments, the visualization catheter shaft is configured to house a camera and a light source.

In certain embodiments, the visualization catheter is configured to house a fiber optic bundle.

In certain embodiments, the visualization catheter shaft is configured to be recessed proximally within the outer catheter shaft.

In certain embodiments, an outer diameter of the inner catheter shaft is approximately equal to an outer diameter of the visualization catheter shaft.

In certain embodiments, the visualization catheter shaft is configured to be inserted into the outer catheter shaft during a first time period, and wherein the visualization catheter shaft is configured to be removed from the outer catheter shaft and replaced with the inner catheter shaft during a second time period.

Other objects, features, and advantages of the present invention will be apparent to one of skill in the art from the following detailed description and figures.

### BRIEF DESCRIPTION OF THE FIGURES

The present application can be understood by reference to the following drawings, wherein like reference numerals represent like elements. The drawings are merely exemplary to illustrate certain features that may be used singularly or in combination with other features and the present application should not be limited to the embodiments shown.

FIG. 1A is a sectional side view of an embodiment of an embryo transfer catheter system in accordance with the present disclosure.

FIG. 1B is cross-sectional view of the embryo transfer catheter system of FIG. 1A.

FIG. 1C is an enlarged sectional side view of the embryo transfer catheter system of FIG. 1A, illustrating an embryo loaded in the embryo transfer catheter.

FIG. 2A is a side sectional view of an embodiment of an embryo transfer catheter system in accordance with the present disclosure.

FIG. 2B is a cross-sectional view of the embryo transfer catheter system of FIG. 2A.

FIG. 3A is a side sectional view of an embodiment of a visualization catheter for use with, or as part of, an embryo transfer catheter of system in accordance with the present disclosure.

FIG. 3B is a cross-sectional view of the visualization catheter of FIG. 3A.

FIG. 4 is an enlarged side sectional view of an embodiment of an embryo transfer catheter system in accordance with the present disclosure, illustrating an embryo loaded in the embryo transfer catheter.

### DETAILED DESCRIPTION

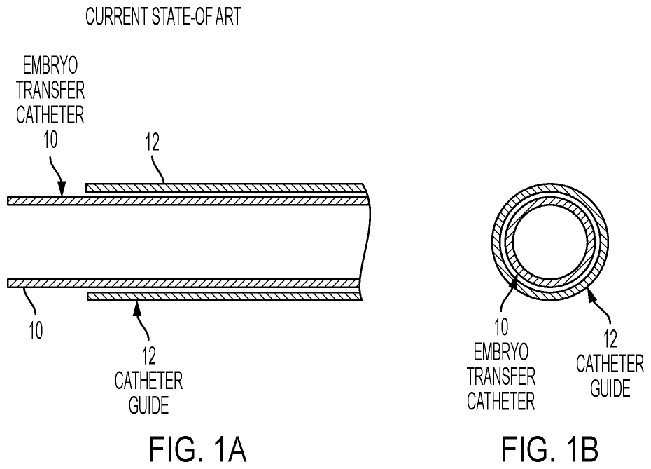

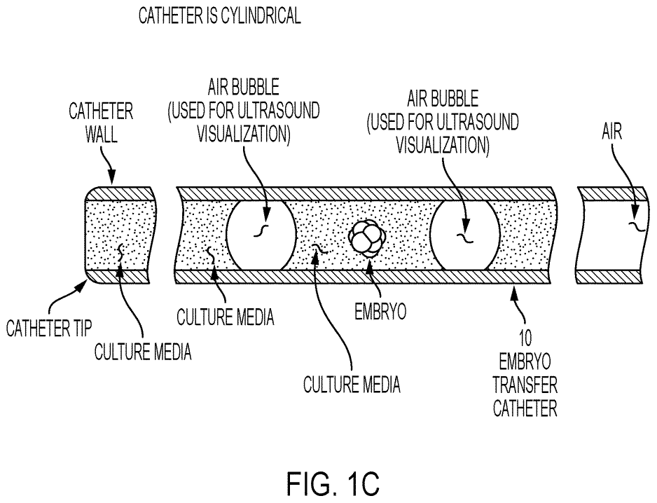

Referring now to the drawings wherein like reference numerals are used to identify like elements in the various views, FIGS. 1A-1C illustrate an exemplary embodiment of a current state-of-the-art embryo transfer catheter system. Current embryo transfer systems include, for example, the Wallace SURE VIEW ® catheter owned by CooperSurgical, Inc., and the catheters described in U.S. Pat. Nos. 6,165,165 and 10,045,756, which are hereby incorporated by reference in their entireties as if fully set forth herein. The system includes an inner catheter or embryo transfer catheter 10 and an outer catheter or guide catheter 12. The embryo transfer catheter 10 is configured to be inserted into the guide catheter 12 (i.e., the guide catheter 12 is larger in diameter than the embryo transfer catheter 10). The embryo transfer catheter 10 is soft and flexible with a soft-radiused distal end. The guide catheter 12 is stiffer than the embryo transfer catheter 10, but still flexible. Both the embryo transfer catheter 10 and the guide catheter 12 include centimeter depth markings which are visible under ultrasound. Further, both the embryo transfer catheter 10 and the guide catheter 12 include hubs fitted onto the proximal ends of their respective catheters. The hubs are designed to mate together when the embryo transfer catheter 10 is inserted into the guide catheter 12. A standard syringe, such as a 1cc syringe, for example, can be mated with the proximal end hub to load an embryo into the embryo transfer catheter 10.

Referring now to FIG. 1C, the embryo transfer catheter 10 is shown with a loaded embryo. The embryo is suspended in culture media and typically sandwiched between two air bubbles, which are used for ultrasound visualization. The proximal end of the embryo transfer catheter 10 includes a relatively large volume of compressed air that has been transferred from the syringe used to load the embryo. The use of a syringe containing compressed air to load the embryo transfer catheter 10 and transfer the embryo to the uterus presents several problems. For example, the barrel of the syringe is typically large compared to the diameter of the catheter. Consequently, a small displacement of the syringe can result in a large displacement of the embryo through the embryo transfer catheter 10 and potentially out from the catheter's distal tip. Further, any twisting or bending of the embryo transfer catheter 10, which often occurs during difficult embryo transfer procedures, can cause occlusion of the catheter. When the syringe is subsequently deployed, transferring a large volume of compressed air along with the embryo, air pressure builds in the embryo transfer catheter 10 and can cause the embryo to be launched (not placed) into the uterus. This results in poor control of the embryo placement procedure, which can lead to inaccurate placement and potentially ectopic implantation.

The embryo transfer catheter system shown in FIGS. 1A-1C is typically used in conjunction with ultrasound visualization. However, IVF patients can have uterine fibroids or polyps that make the uterine wall less receptive to embryo implantation. These fibroids and polyps are not visible under ultrasound, which diminishes the value of ultrasound visualization. Alternative visualization methods, such as the use of an endoscope in conjunction with carbon dioxide or saline to expand the tissue, are not possible because of the damage they could cause to the embryo. Thus, the target location for embryos is typically the most receptive uterine location, which is about 1 cm below the top of the uterus, not far from the fallopian tubes.

Furthermore, the catheter system shown in FIGS. 1A-1C is often used together with a malleable stylet or wire (not shown). The malleable stylet typically has the same or similar diameter as the embryo transfer catheter 10, and it fits inside the guide catheter 12. The malleable stylet is often used for “difficult transfers” based on geometry or the anatomy of the patient. For example, a physician may bend the stylet in the shape they believe will match the patient's anatomy. The bent stylet is inserted into the guide catheter 12 and then placed into the patient, after which the stylet can be removed and replaced with the embryo transfer catheter 10. Again, this practice often results in poor control of the catheter system and inaccurate embryo placement.

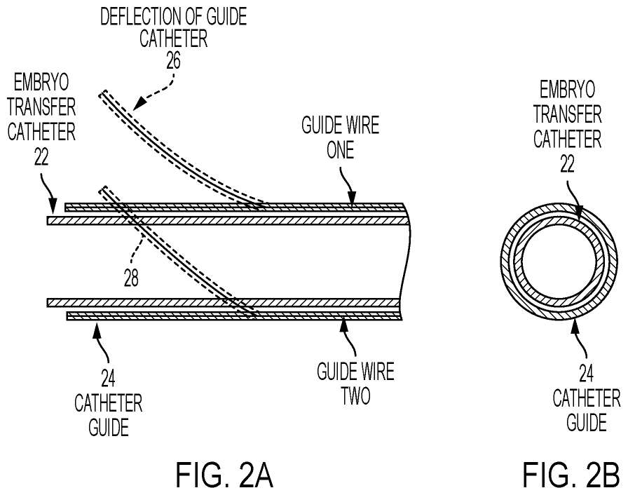

Referring now to FIGS. 2A-2B, an embodiment of an improved embryo transfer catheter system is shown. The improved embryo transfer catheter system is similar to the current state-of-the-art catheter system described in FIGS. 1A-1C in that it includes an inner catheter or embryo transfer catheter 22 and an outer catheter or guide catheter 24. The embryo transfer catheter 22 is configured to be inserted into the guide catheter 24 (i.e., the guide catheter 24 is larger in diameter than the embryo transfer catheter 22). The embryo transfer catheter 22 is soft and flexible with a soft-radiused distal end. The guide catheter 24 is stiffer than the embryo transfer catheter 22, but still flexible. Both the embryo transfer catheter 22 and the guide catheter 24 can include centimeter depth markings that are visible under ultrasound, as well as proximal end hubs designed to mate together and/or with a plunger, piston, or syringe, for example. Both catheters 22 and 24 can be disposable or single-use catheters.

The improved embryo transfer catheter system differs from the current state-of-the-art embryo transfer catheter system in several key aspects. First, as shown in FIG. 2A, the guide catheter 24 is steerable and deflectable, as it includes two pull wires (also known as deflection wires or guide wires) 26 and 28 embedded within its wall. The pull wires can be conventional pull wires used in steerable catheters. In some embodiments, the guide catheter can include a different number of pull wires, such as one pull wire, three pull wires, or four pull wires, for example. Pull wires 26 and 28 can be located 180 degrees apart, as shown in FIG. 2A, or they can be located at any location within the wall of guide catheter 24. For example, pull wires 26 and 28 can be positioned 90 degrees or 270 degrees apart within the catheter wall. Pull wires 26 and 28 can be controlled via a lever or wheel. For example, pushing a lever or rotating a wheel coupled to the pull wires can pull one wire and release the opposite wire, causing deflection of the catheter tip. Alternatively, in a four-wire system, for example, a joy stick coupled to one or more rotational elements, which are in turn coupled to the pull wires, can be used to control deflection. The use of pull wires in guide catheter 24 could eliminate the current practice of using a malleable stylet, as described above with respect the current state-of-the art catheter system shown in FIGS. 1A-1C.

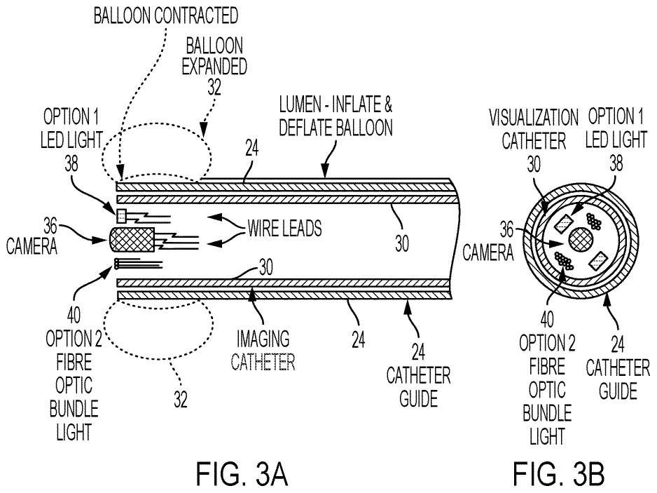

The improved embryo transfer catheter system also differs from the current state-of-the-art embryo transfer catheter system in terms of visualization. As shown in FIGS. 3A-3B, the improved embryo transfer catheter system can include a visualization (or imaging) catheter 30 configured to be inserted into the guide catheter 24. The visualization catheter 30 can be disposable, and it can be similar in diameter to the embryo transfer catheter 22. The visualization catheter 30 can be inserted into the guide catheter 24 first, and then removed and replaced with the embryo transfer catheter 22 once the target transfer location has been confirmed. As shown in FIG. 3A, a balloon 32 can be used in conjunction with the visualization catheter to expand the surrounding tissue for improved visualization. A lumen for inflation and deflation of the balloon can extend back to the catheter handle, where air can be pumped into or withdrawn from the balloon. The balloon material can comprise silicone or silicone-like material. The balloon material can be bonded to the catheter tip.

The visualization catheter 30 can include one or more visualization mechanisms and corresponding imaging tools. For example, the visualization catheter 30 can include a camera 36 and at least one LED light 38 embedded within the tip of the catheter. Alternatively, a fiber optic bundle 40 embedded withing the tip of the catheter can be used in conjunction with a camera and light source that are located elsewhere. The optical fibers can be used to transmit light and images to and from the catheter tip, respectively. Although FIG. 3A shows the distal end of the visualization catheter 30 in flush with the distal end of the guide catheter 24, the distal end of the guide catheter 24 can be extend further distally to provide the camera 36 with a focal length to obtain clear images of the surrounding uterine tissue. For example, the visualization catheter 30 can be proximally recessed into the guide catheter 24 approximately 1 cm or more to allow for identification and imaging of the target transfer location. The visualization catheter 30 can be used in place of, or in addition to, the current practice of ultrasound visualization.

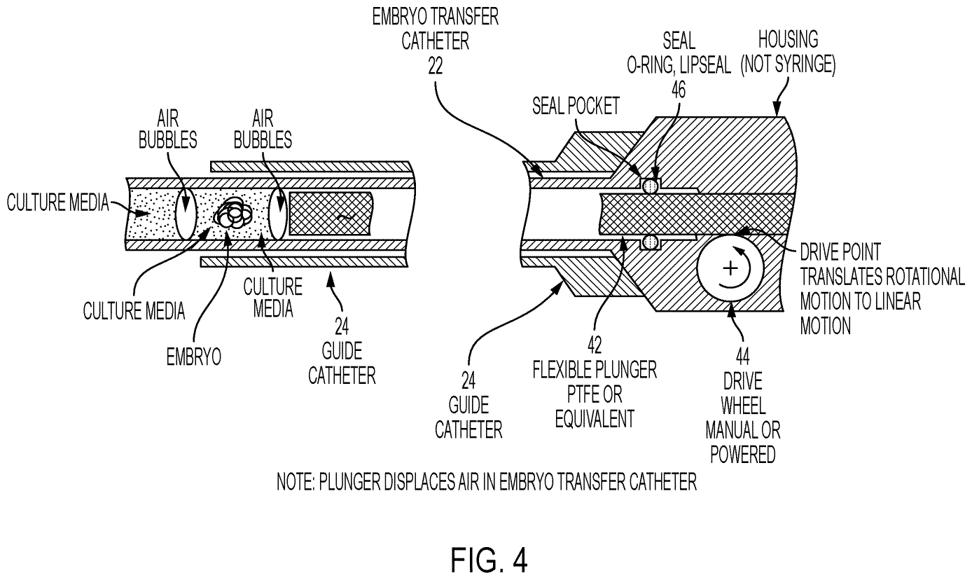

Referring now to FIG. 4, another way in which the improved embryo transfer catheter system differs from the current state-of-the-art embryo transfer catheter system is the inclusion of a plunger (or piston or rod) 42 within the lumen of the embryo transfer catheter 22. The plunger 42 can allow for controlled delivery of the embryo from the distal end of the catheter 22. The plunger 42 can be made from polytetrafluoroethylene (PTFE or TEFLON®) or another flexible and biocompatible material. The plunger 42 can be integral to the embryo transfer catheter 22, and it can be attached or anchored to the proximal end housing of the catheter. The plunger 42 can extend throughout the length of the lumen of the embryo transfer catheter 22, and it can be slightly smaller in diameter than the catheter. The plunger 42 can move linearly along the longitudinal axis (i.e., proximally and distally) within the lumen of the embryo transfer catheter 22. In an embodiment, movement of the plunger 42 can be controlled, either manually or electronically, via a drive wheel 44, as shown in FIG. 4. A friction element between the drive wheel 44 and the plunger 42 (e.g., gears) can allow for the translation of rotational movement to linear movement. In other embodiments, movement of the plunger 42 can be controlled via other means, such as a thumb wheel or knob located on the handle or housing of the catheter or a direct pushing of the proximal end of the plunger 42. Movement of the plunger 42 can also be controlled electronically (e.g., by a motor) or robotically, for example. It should be noted that a seal between the plunger 42 and the embryo transfer catheter 22 is needed to provide a vacuum when the catheter is loaded (e.g., when an embryologist draws the embryo into the catheter). The location of the seal differentiates the plunger 42 from a typical syringe plunger, where the seal is larger and formed along the length of the plunger. As shown in FIG. 4, an O-ring seal 46 can be used for this purpose; however, this is merely an example of various types sealing mechanisms that can be used.

An advantage of the using the plunger 42, as opposed to a traditional syringe, to load and transfer the embryo from the catheter 22, is that movement of the plunger 42 within the lumen of the catheter 22 provides an approximately 1:1 ratio of motion-in other words, moving the plunger distally 1 cm would push the loaded embryo approximately 1 cm distally along or out of the catheter lumen. In contrast, a distal movement of a traditional syringe by 1 cm would result in much greater distal movement of the embryo within (and out from the distal end of) the catheter. This is because the volume of a traditional 1 cc syringe can be approximately fifty times the volume of an embryo transfer catheter. Thus, the plunger 42 provides positive displacement of the catheter contents, resulting in an approximately 1:1 ratio of motion between the plunger itself and the contents of the catheter (i.e., the embryo suspended in culture media). In this way, the plunger 42 eliminates or greatly reduces that large mechanical advantage of a traditional syringe piston. Moreover, the volume of fluid displaced by the plunger within the lumen of the catheter 22 is approximately equal to the volume of fluid expelled from the distal end of the catheter 22. This allows the operator of the embryo transfer catheter 22 to have much greater control of embryo placement at the target location and the velocity with the embryo is released. (It should be noted, however, that precise 1:1 motion may not be achievable, as the small space between the outer diameter of the plunger 42 and the inner diameter of the catheter embryo transfer 22 can introduce small errors.)

Another advantage of using the plunger 42, as opposed to a traditional syringe, to push the embryo out from the distal end of the embryo transfer catheter 22 is that it eliminates or greatly reduces the amount of air within the catheter 22. During difficult embryo transfer procedures, embryo transfer catheters used with a traditional syringe can become twisted, bent, or kinked, causing a temporary occlusion of the catheter lumen. This results in compressed air building up proximally or distally from the occlusion. When the catheter straightens and the occlusion resolves, built up compressed air can launch the embryo out of the distal end of catheter at a high velocity and in an uncontrolled manner. In contrast, the relatively small amount of air within catheter 22, permitted by the design of plunger 42 as described above, can reduce or eliminate the likelihood of compressed air building up within the catheter. While small air bubbles may surround the non-compressible culture media in which the embryo is suspended, the overall amount of air in the embryo transfer catheter 22 is greatly reduced via use of the plunger 42. Consequently, decreased pressure within the catheter 22 and improved control of the of embryo release allows for more accurate and precise placement within the uterus.

Yet another advantage of the present improved embryo transfer catheter system is that it can be robotically implemented. A robotically-controlled or automated guide catheter can provide improved catheter visualization, control, and navigation as compared to the current state-of-the-art catheters. Improved visualization can allow physicians to better see where the embryo is being implanted in the uterus. Because the surface and receptiveness of the uterine wall changes continually throughout a woman's menstrual cycle, improved visualization can allow for ideal placement of the embryo based on the condition of the uterine wall. For example, improved visualization can help avoid placing the embryo in the wrong location such as on a polyp, fibroid, or in the fallopian tube. Visualization can also facilitate viewing of the follicles on the uterine wall to make sure that they are in the receptive phase. Finally, visualization can allow the physician or embryologist to be able to see the embryo after placement within the uterus.

Robotic implementation of the embryo transfer catheter system disclosed herein can also provide improved accuracy and precision in motion control, rates of dispensation, and the like. Robotic or automated features of the guide catheter allow for superior guidance and navigation in comparison to the current manual control process used with existing guide catheters. For example, in cases of complex uterine geometry (e.g., a retroverted, retroflexed, or anteverted uterus), robotic control can allow for manipulation of tortuous pathways without damaging sensitive tissue. Because the guide catheter disclosed herein can deflect and bend, it can be inserted and guided through difficult uterine geometry with the camera visualizing the process and pathway. The guidance and visualization can provide a feedback loop with the camera, allowing the physician or embryologist to confirm that the guide catheter is on the correct course or has arrived at the correct location. The camera can be used to aid in correcting the course of the catheter and/or avoiding potential problem areas. Improved guidance and navigation of the guide catheter allows for enhanced accuracy and precision when inserting the embryo transfer catheter.

Although at least one embodiment of an embryo transfer catheter system has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment,” or the like, means that a particular feature, structure, or characteristic described in connection with the embodiment is included in at least one embodiment. Thus, appearances of the phrases “in various embodiments,” “in some embodiments,” “in one embodiment,” or “in an embodiment,”or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The terms “about” and “approximately” may be used throughout the specification when referring to a measurable value, such as an amount, a distance, a temporal duration, and the like. The terms “about” and “approximately” are meant to encompass variations of ±20% or ±10%, in certain embodiments ±5%, in certain embodiments ±1%, in certain embodiments ±0.1% from the specified value, as such variations are appropriate in accordance with the present disclosure.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter assembly comprising:
an elongated inner catheter shaft comprising a proximal end, a distal end, and a central lumen; and
an axially movable plunger extending at least partially within the central lumen;
wherein the plunger is configured to axially move a first distance within the central lumen and to positively displace a volume of a fluid in the central lumen by about the first distance.

2. The catheter assembly of claim 1, further comprising a seal between the plunger and the central lumen.

3. The catheter assembly of claim 2, wherein the plunger is configured to move proximally to draw fluid from outside the inner catheter shaft through the distal end and into the central lumen.

4. The catheter assembly of claim 1, wherein the fluid is non-compressible.

5. The catheter assembly of claim 4, wherein the fluid is culture media comprising an embryo.

6. The catheter assembly of claim 5, wherein the plunger is configured to move distally to transfer the embryo from the central lumen out through the distal end of inner catheter shaft.

7. The catheter assembly of claim 5, wherein the plunger is configured to move distally to transfer the embryo from the distal end of the inner catheter shaft to a target location within a uterine cavity.

8. The catheter assembly of claim 1, wherein an outer diameter of the plunger is approximately equal to an inner diameter of the central lumen.

9. The catheter assembly of claim 1, wherein the plunger comprises PTFE.

10. The catheter assembly of claim 1, further comprising a drive wheel frictionally mated with the plunger; wherein rotational movement of the drive wheel translates to linear movement of the plunger.

11. The catheter assembly of claim 10, wherein the drive wheel is configured to be operated manually, via a motor, or robotically.

12. The catheter assembly of claim 1, further comprising one or more markers visible via ultrasound.

13. The catheter assembly of claim 1, further comprising an elongated outer catheter shaft, wherein the inner catheter shaft is configured to be inserted into the outer catheter shaft.

14. The catheter assembly of claim 13, further comprising at least one pull wire extending within a wall of the outer catheter shaft from a proximal end of the outer catheter shaft to a distal end of the outer catheter shaft; wherein the at least one pull wire is configured to deflect the distal end of the outer catheter shaft.

15. The catheter assembly of claim 13, further comprising at least one expandable balloon axially mounted to a distal end of the outer catheter shaft.

16. The catheter assembly of claim 13, further comprising a visualization catheter shaft configured to be inserted into the outer catheter shaft.

17. The catheter assembly of claim 16, wherein the visualization catheter shaft is configured to house a camera and a light source.

18. The catheter assembly of claim 16, wherein the visualization catheter is configured to house a fiber optic bundle.

19. The catheter of claim 16, wherein the visualization catheter shaft is configured to be recessed proximally within the outer catheter shaft.

20. The catheter assembly of claim 16, wherein an outer diameter of the inner catheter shaft is approximately equal to an outer diameter of the visualization catheter shaft.

21. The catheter assembly of claim 16, wherein the visualization catheter shaft is configured to be inserted into the outer catheter shaft during a first time period, and wherein the visualization catheter shaft is configured to be removed from the outer catheter shaft and replaced with the inner catheter shaft during a second time period.
